(19) United States
(12) Patent Application Publication
(10) Pub. No.: US 2025/0372203 A1
(43) Pub. Date: Dec. 4, 2025

(54) Identifying Peptides with Multiple Post-Translational Modifications Using Mixed Integer Linear Programming

(71) Applicant: The Hong Kong University of Science and Technology, Hong Kong (CN)

(72) Inventors: Weichuan YU, Hong Kong (CN); Ning LI, Hong Kong (CN); Shengzhi LAI, Hong Kong (CN)

(21) Appl. No.: 19/194,567

(22) Filed: Apr. 30, 2025

Related U.S. Application Data
(60) Provisional application No. 63/652,689, filed on May 29, 2024.

(51) Int. Cl. G16B 30/00; G16B 40/20

(52) U.S. Cl. CPC G16B 30/00; G16B 40/20

(57) ABSTRACT

Identifying peptides with multiple post-translational modifications (PTMs) by tandem mass spectrometry (MS2) is computationally challenging since it involves finding the optimal PTM pattern that produces theoretical spectra most closely resembling experimental spectra. To address this issue, a mixed integer linear programming (MILP) model is used to find an optimal solution to peptide identification and PTM characterization. The optimal solution is integrated into a tool named as PIPI3. PIPI3 identifies the optimal PTM pattern without enumerating all possible PTM combinations. On simulation datasets with up to four PTMs per peptide, PIPI3 correctly identified over 99% of the spectra and characterized the PTM patterns with a precision of 85%, while the numbers of the best competitor MODplus are 92% and 76%, highlighting PIPI3's advantage in handling peptides with multiple PTMs compared to state-of-the-art techniques.

### CROSS-REFERENCE TO RELATED APPLICATIONS

This application claims priority to, and the benefit of, U.S. Provisional Patent Application Ser. No. 63/652,689 filed May 29, 2024, the disclosure of which is incorporated by reference herein in its entirety.

### ABBREVIATIONS

FM-index full-text index in minute space
FDR false discovery rate
MILP mixed integer linear programming
MS mass spectrometry
MS2 tandem mass spectrometry
PSM peptide-spectral match
PTM post-translational modification
PWC piece-wise constant

### TECHNICAL FIELD

The present disclosure generally relates to protein sequencing. Particularly, the present disclosure relates to analyzing a protein sample with MS2 for protein sequencing, where the protein sample is probable of having multiple PTMs.

### BACKGROUND

PTMs are essential in the regulation of cellular functions. Over the past three decades, extensive research efforts have focused on elucidating the regulatory mechanisms of PTMs with the assistance of MS [1]. Subsequently, increased attention has been directed towards understanding the combinatorial effects of multiple PTMs, collectively referred to as PTM crosstalk [2], which can exert positive or negative influences on each other. Notably, certain PTM crosstalk events may occur within the same protein, or even within the same peptide [3], highlighting the need for efficient software for peptides with multiple PTMs without prior specification.

However, it remains challenging to identify peptides with multiple PTMs using existing database search methods, due to the exponential growth of database sizes with respect to the number of predetermined PTMs. Since 2010s, some methods have been developed to identify peptides with PTMs from PTM databases (such as UNIMOD [4]). Based on the difference between the mass of the peptide candidate and the precursor mass, alignment and enumeration strategies have been employed to characterize the PTM patterns, i.e. the numbers, masses, and sites of PTMs in the peptides. Most of these methods use short amino acid sequences, called tags, extracted from experimental peaks. Concretely, PeaksPTM [5] exhaustively enumerates peptides with common PTMs or unspecified PTMs individually and subsequently combines them to detect multiple PTMs in peptides. MODplus [6] and PIPI [7] utilize dynamic programming to align extracted tags with peptide sequences to characterize PTMs. Open-pFind [8] characterizes PTMs by enumerating all single PTMs or two-PTM combinations. TagGraph [9] also employs dynamic programming to align potentially PTM-containing candidates with de novo sequences to localize multiple PTMs on peptide sequences. MSFragger enumerates all possible modified locations using regular ion matched peaks and shifted ion matched peaks to localize PTMs. However, these methods exhibit unsatisfactory performance when identifying peptides with multiple PTMs.

There is a need in the art for a technique offering an improved performance in identifying peptides with multiple PTMs.

### SUMMARY

A first aspect of the present disclosure is to provide a method for identifying a plurality of peptides from a protein sample. An individual peptide in the plurality of peptides is probable of having multiple PTMs.

The method comprises the steps of: (a) obtaining a tandem mass spectra dataset obtained for the sample; (b) extracting tags from a weighted directed graph formed according to spectral peaks identified in the dataset; (c) using the extracted tags to retrieve a plurality of protein candidates potentially present in the sample from a FM-indexed protein database; (d) segmenting an individual protein candidate into a plurality of sections according to locations of the extracted tags resided in the individual protein candidate, wherein the plurality of sections consists of a N-section, a C-section and a gap section; (c) optimizing a MILP model that models an individual section of the individual protein candidate to yield a peptide backbone and a PTM pattern for the individual section, wherein the MILP model is optimized under an objective of maximizing a total intensity of matched spectral peaks as matched by b- and y-ions in the individual section; (f) repeating the steps (d) and (e) until the plurality of protein candidates is processed; and (g) forming the plurality of peptides according to respective peptide backbones and respective PTM patterns obtained for the plurality of protein candidates.

Preferably, the method further comprises the step (h) of using a target-decoy strategy to assess quality of the plurality of peptides obtained in the step (g).

In certain embodiments, a FPR is computed for the individual peptide according to the target-decoy strategy in the step (h).

In certain embodiments, the plurality of protein candidates is identified by using a fuzzy and bidirectional match strategy to match the extracted tags in respective peptide sequences stored in the FM-indexed protein database in the step (c).

In certain embodiments, the spectral peaks in the dataset are first identified according to a tandem mass spectral database in the step (b).

In certain embodiments, the MILP model is optimized by Gurobi Optimizer in the step (e).

In certain embodiments, the weighted directed graph is formed by calculating nodes and edges of the weighted directed graph according to the spectral peaks and potential amino acids that lie between successive peaks in the step (b).

In certain embodiments, the plurality of protein candidates is retrieved by using the extracted tags having various lengths in the step (c).

In certain embodiments, a FM-index used in the FM-indexed protein database consists of a forward index and a backward index such that bidirectional searches are supported.

In certain embodiments, the FM-index is modified to enable a fuzzy search.

In certain embodiments, amino acids in either the N-section or the C-section are grouped into fixed ones, optional ones and infeasible ones in the step (d).

In certain embodiments, constraints in the MILP model involve all fixed and optional amino acids, and all possible PTMs on every amino acid as recorded in the UNIMOD database.

A second aspect of the present disclosure is to provide a system for identifying a plurality of peptides from a protein sample. An individual peptide in the plurality of peptides is probable of having multiple PTMs.

The system comprises one or more computers configured to execute a process of identifying the plurality of peptides from the protein sample according to any of the embodiments of the method disclosed above.

In certain embodiments, the system further comprises a tandem mass spectrometer for analyzing the sample with tandem mass spectroscopy to thereby generate the tandem mass spectra dataset obtained for the sample.

Other aspects of the present disclosure are disclosed as illustrated by the embodiments hereinafter.

### BRIEF DESCRIPTION OF THE DRAWINGS

FIG. 1 depicts a workflow illustrating exemplary steps of a method as disclosed herein for identifying a plurality of peptides from a protein sample, where an individual peptide in the plurality of peptides is probable of having multiple PTMs.

FIG. 2 provides an illustrative example of different steps used in the disclosed method.

FIG. 3, which includes subplots A-C, shows performance results of PIPI3 against existing technique, where: subplot A shows the numbers of PSMs with correct backbones and the number of PSMs with correct PTM patterns; subplot B shows the sensitivity of PTM characterization; and subplot C shows the sensitivity of each individual PTM.

Skilled artisans will appreciate that elements in the figures are illustrated for simplicity and clarity and have not necessarily been depicted to scale.

### DETAILED DESCRIPTION

Regarding the terminology used herein, it is intended that the present disclosure adopts commonly-accepted interpretation of technical terms used in the fields of MS and of peptide sequencing. “A b-ion” of a peptide is a charged fragment of the peptide after the peptide is split at a peptide bond between two constituent amino acids, where the b-ion contains the N-terminus of the peptide. “A y-ion” of a peptide is a charged fragment of the peptide after the peptide is split at a peptide bond between two constituent amino acids, where the y-ion contains the C-terminus of the peptide. “A tag” is a partial sequence of an amino-acid sequence where the partial sequence is formed by consecutive amino acids.

The present disclosure is concerned with PTM characterization. From a combinatorial perspective, the PTM characterization problem can be viewed as identifying the optimal PTM combinations on the peptide backbone sequence, with which the theoretically generated MS2 spectrum is most similar to the experimental MS2 spectrum. Due to the enormous number of potential PTM combinations, existing methods can only resort to seeking approximate solutions. These limitations have underscored the necessity for a more profound understanding of the PTM characterization problem, intending to find the optimal solution to the identification of peptides with multiple PTMs.

The Inventors have developed PIPI3, a novel optimization-based technique that advantageously utilizes a MILP model for finding an optimal solution to the identification of peptides with multiple PTMs. PIPI3 uses a fuzzy and bidirectional matching approach to locate tags on protein sequences. Subsequently, protein sequences with located tags are selected as candidates. PIPI3 then constructs and solves a tag-based MILP model to simultaneously determine the peptide backbone and the PTM pattern. Advantageously, PIPI3 identifies the optimal PTM pattern without enumerating all possible PTM combinations. It reduces the amount of required computation in comparison to an existing, commonly-used approach of considering all PTM combinations. To assess the performance of PIPI3 in backbone identification and PTM characterization, the Inventors have conducted extensive validations using different datasets and compared the results with those obtained from two state-of-the-art methods. Moreover, the Inventors have applied PIPI3 to identify PTM combinations that were not detected by other software programs.

In PIPI3, the raw file of MS2 spectra undergoes pre-processing using MSConverter [11], and tags are extracted from the MS2 spectra. These tags are subsequently used to retrieve candidates from the FM-indexed protein database with a fuzzy and bidirectional matching approach. The mass difference between a peptide sequence and the precursor of an MS2 spectrum, denoted as Δm, is considered as the total mass of potential PTMs, which are then characterized using MILP models and solved by Gurobi [13]. Subsequently, peptide candidates with characterized PTM patterns are collected and reranked by the protein feedback module [14]. Finally, PIPI3 outputs a PSM list with FDR controlled by the target-decoy strategy [15].

Embodiments of the present disclosure are developed and elaborated as follows based on details of PIPI3.

A first aspect of the present disclosure is to provide a method for identifying a plurality of peptides from a protein sample. Each peptide in the plurality of peptides is probable of having multiple PTMs. More specifically, each peptide may have any number of PTMs, including special cases of having no PTM and of having a single PTM.

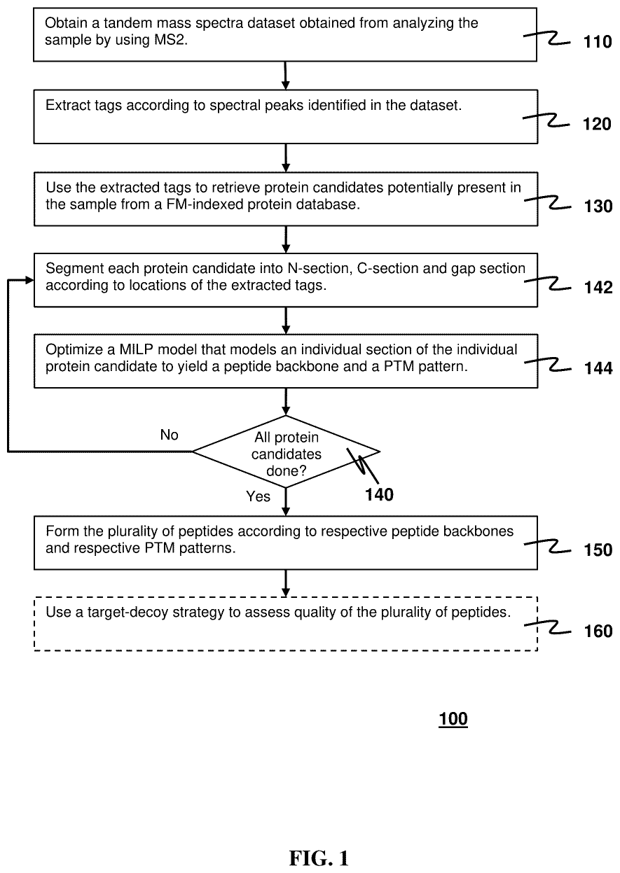

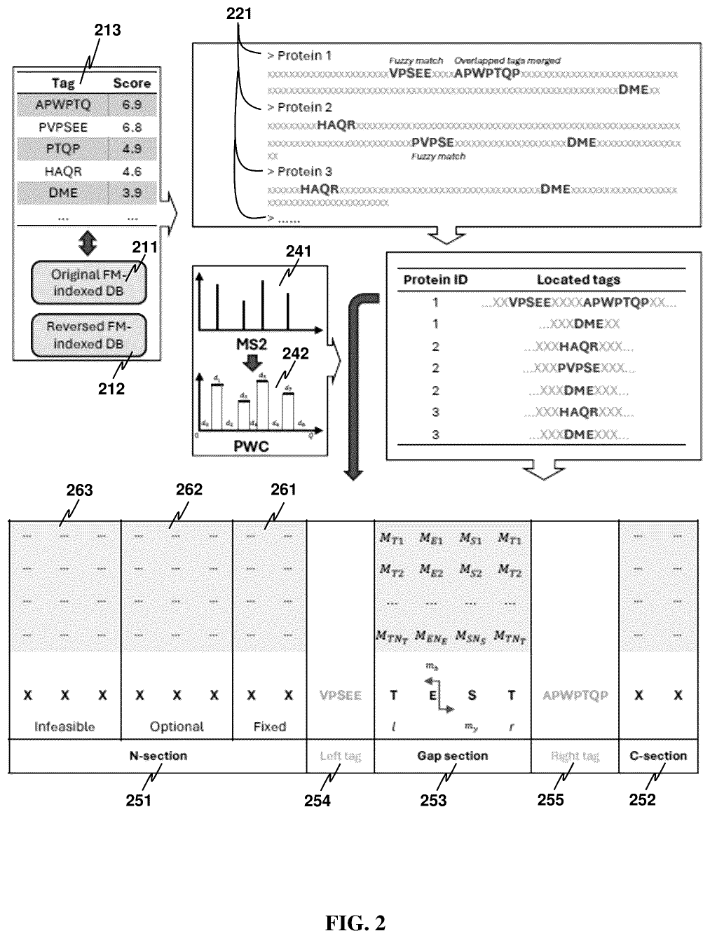

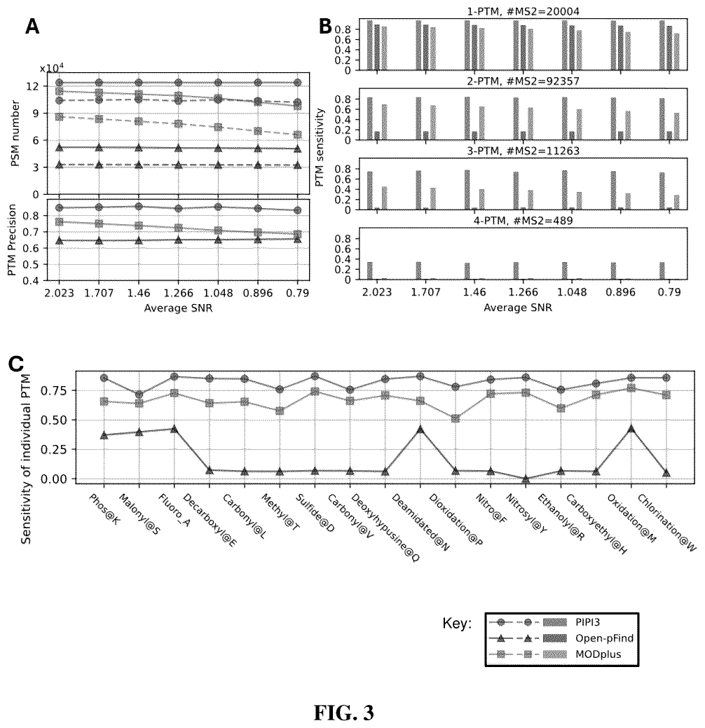

The disclosed method is exemplarily illustrated with the aid of FIGS. 1 and 2. FIG. 1 depicts a workflow 100 illustrating exemplary steps of the disclosed method. FIG. 2 provides an illustrative example of different steps used in the disclosed method. Exemplarily, the workflow 100 for identifying the plurality of peptides from the protein sample comprises steps 110, 120, 130, 140, 142, 144 and 150.

The step 110 may be regarded as an initialization step. In the step 110, a tandem mass spectra dataset obtained for the sample is obtained. The dataset consists of one or more tandem mass spectra 241 obtained by using MS2 to analyze the sample.

In the step 120, tags 213 are extracted from a weighted directed graph that is formed according to spectral peaks identified in the dataset. Generally, the weighted directed graph is formed by calculating nodes and edges of the weighted directed graph according to the spectral peaks and potential amino acids that lie between successive peaks. The spectral peaks in the dataset may first be identified according to a tandem mass spectral database.

After the tags 213 are extracted, the extracted tags 213 are used in the step 130 to retrieve a plurality of protein candidates 221 potentially present in the sample from a FM-indexed protein database. Generally, the plurality of protein candidates 221 is retrieved by using the extracted tags 213 having various lengths. It is preferable that the plurality of protein candidates 221 is identified by using a fuzzy and bidirectional match strategy to match the extracted tags 213 in respective peptide sequences stored in the FM-indexed protein database. It is also preferable that a FM-index used in the FM-indexed protein database consists of a forward index (viz, an index used in an original FM-indexed database 211) and a backward index (viz., an index used in a reverse FM-indexed database 212) such that bidirectional searches are supported. Preferably and advantageously, the FM-index is modified to enable a fuzzy search.

In the step 140, the steps 142 and 144 are repeated until the plurality of protein candidates is processed. That is, the steps 142 and 144 are repeated until execution of these two steps for respective protein candidates in the plurality of protein candidates is done.

In the step 142, an individual protein candidate in the plurality of protein candidates is segmented into a plurality of sections according to locations of the extracted tags resided in the individual protein candidate. The plurality of sections consists of a N-section 251, a C-section 252 and a gap section 253. The gap section 253 is a first portion of the individual protein candidate between two adjacent tags, referred to as a left tag 254 and a right tag 255. The N-section 251 is a second portion of the individual protein candidate from the left tag 254 towards an N-terminus. The C-section 252 is a third portion of the individual protein candidate from the right tag 255 towards a C-terminus. In certain embodiments, amino acids in either the N-section 251 or the C-section 252 are grouped into fixed ones 261, optional ones 262 and infeasible ones 263.

In the step 144, a MILP model that models an individual section of the aforesaid individual protein candidate as used in the step 142 is optimized to yield a peptide backbone and a PTM pattern for the individual section. The peptide backbone and PTM pattern constitute a PSM. The individual section is selected from the plurality of sections of the aforesaid individual protein candidate. The MILP model is optimized under an objective of maximizing a total intensity of matched spectral peaks as matched by b- and y-ions in the individual section. Construction and optimization of the MILP model is further explained as follows.

Consider a simpler case where the sequence within the gap section 253 is fixed and ΔM can be easily computed as the mass difference between the start peak of the right tag 255 and the end peak of the left tag 254, i.e. ΔM=Mᵣ−Mₗ. Each PTM on the amino acids in the gap section 253 is assigned a binary variable x. By imposing constraints on the total number of PTMs and the number of PTMs on the amino acids, m_{b} and m_{y} can be expressed as linear combinations of the constants and variables. The objective is to maximize the total intensity of the matched experimental peaks by all m_{b} and m_{y}. The model is formulated as follows:

$${max}_{x} {\sum }_{b - ions} P W C ( {m}_{b} ) + {\sum }_{y - ions} P W C ( {m}_{y} ) - 0.1 {\sum }_{a ∈ [ l , r ]} {\sum }_{p ∈ [ 0 , {N}_{a} ]} {x}_{p} \qquad ( 1 )$$

$$s . t . \vert {\sum }_{a ∈ [ l , r ]} {\sum }_{p ∈ [ 0 , {N}_{a} ]} {x}_{p} {M}_{p} - Δ M \vert \leq {τ}_{1} , \qquad ( 2 )$$

$${\sum }_{a ∈ [ l , r ]} {\sum }_{p ∈ [ 0 , {N}_{a} ]} {x}_{p} \leq {P}_{max} , \qquad ( 3 )$$

$${\sum }_{p ∈ [ 0 , {N}_{a} ]} {x}_{p} \leq 1 , ∀ a , \qquad ( 4 )$$

$${m}_{b} = {M}_{l} + {\sum }_{a ∈ [ l , b ]} {M}_{a} + {\sum }_{a ∈ [ l , b ]} {\sum }_{p ∈ [ 0 , {N}_{a} ]} {x}_{p} {M}_{p} , ∀ b ∈ [ l , r ] , \qquad ( 5 )$$

$${m}_{y} = {M}_{r} + {\sum }_{a ∈ [ y , r ]} {M}_{a} + {\sum }_{a ∈ [ y , r ]} {\sum }_{p ∈ [ 0 , {N}_{a} ]} {x}_{p} {M}_{p} , ∀ y ∈ [ l , r ] , \qquad ( 6 )$$

$$\vert {m}_{b} + {m}_{y} - C \vert \leq {τ}_{1} , ∀ b , y , b = y , \qquad ( 7 )$$

$$x ∈ \{ 0 , 1 \} , ∀ x . \qquad ( 8 )$$

Equation (1) indicates the objective function that is maximized over x. Constraint (2) enforces that the difference between the total mass of PTMs and ΔM is within τ₁. Constraint (3) enforces the total number of PTMs to be smaller than a given parameter. Constraint (4) enforces that at most one PTM can occur on each amino acid. Constraint (5) and (6) express m_{b} and m_{y} by using other variables. Constraint (7) reduces the feasible zone. Constraint (8) enforces the integrality of x.

The objective function is the summation of the intensities of peaks matched by b and y ions, plus a penalty term on the total number of PTMs. PWC(m) is a PWC function 242 representing the MS2 spectra 241. If the input mass m matches the mass of any peak e, the function returns the corresponding Iₑ; otherwise, it returns 0.

The PWC function 242 is not inherently linear, making it incompatible with a MILP formulation. However, it can be linearized using auxiliary variables. Define binary variables [d₀, d₁, . . . , d₂ₑ] as shown below, and a continuous variable f for the return value of PWC(m). Then, apply for constraints as follows:

$${\sum }_{i = 0}^{2 E} {d}_{i} = 1 , \qquad ( 9 )$$

$$m \geq {\sum }_{e = 1}^{E} ( {M}_{e} - {τ}_{2} + ϵ ) {d}_{2 e - 1} + {\sum }_{e = 1}^{E} ( {M}_{e} + {τ}_{2} + ϵ ) {d}_{2 e} + 0 {d}_{0} , \qquad ( 10 )$$

$$m \leq {\sum }_{e = 1}^{E} ( {M}_{e} - {τ}_{2} ) {d}_{2 e - 2} + {\sum }_{e = 1}^{E} ( {M}_{e} + {τ}_{2} ) {d}_{2 e - 1} + Q {d}_{2 E} , \qquad ( 11 )$$

$$f \leq {F}_{i} + {G}_{i} ( 1 - {d}_{i} ) , ∀ i ∈ [ 0 , 2 E ] , \qquad ( 12 )$$

$$and$$

$${d}_{i} ∈ \{ 0 , 1 \} , ∀ i ∈ [ 0 , 2 E ] , \qquad ( 13 )$$

where the constant Q upper bounds all Mₑ, Fᵢ is the value of each piece (including zeros), Gᵢ is a big constant such that Fᵢ+Gᵢ≥Gⱼ, ∀j≠i, and ϵ is a tolerance constant. The constraints enforce that only one piece can be selected, and M₌ᵢ₋₁≤m≤Mᵢ⇒dᵢ=1. An upper bound is imposed on the return value f, restricting it to Fᵢ only when dᵢ=1. Other inequalities where dᵢ=0 become redundant because of Gᵢ. Since the objective function is maximum-wise, f automatically assumes the value of Fᵢ. As a result, the objective function can be linearized as

$${max}_{x} {\sum }_{b - ions} {f}_{b} + {\sum }_{y - ions} {f}_{y} - 0.1 {\sum }_{a ∈ [ l , r ]} {\sum }_{p ∈ [ 0 , {N}_{a} ]} {x}_{p} .$$

Preferably, constraints in the MILP model involve all fixed and optional amino acids, and all possible PTMs on every amino acid as recorded in the UNIMOD database.

The MILP model may be optimized by Gurobi Optimizer. Other constrained-optimization algorithms may also be used to optimize the MILP model.

After the step 140 is done, the plurality of peptides is formed in the step 150 according to respective peptide backbones and respective PTM patterns obtained for the plurality of protein candidates. Particularly, an individual peptide is formed by concatenating the N-section 251, the left tag 254, the gap section 253, the right tag 255 and the C-section 252 after the respective peptide backbones and respective PTM patterns are optimally determined.

It is preferable and advantageous to assess quality of the plurality of peptides as obtained in the step 150. Preferably and advantageously, the workflow 100 further comprises a step 160 of using a target-decoy strategy to assess quality of the plurality of peptides. In certain embodiments, a FPR is computed for the individual peptide according to the target-decoy strategy.

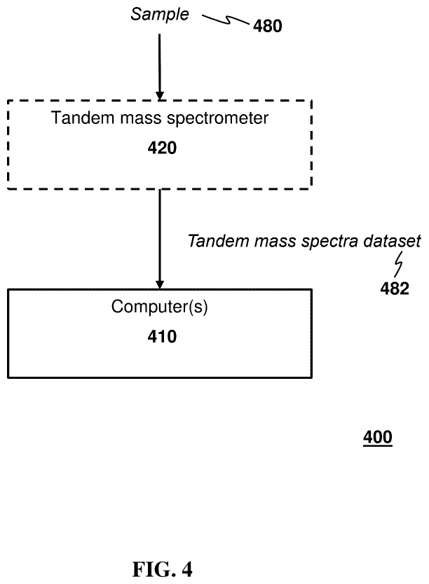

FIG. 3, which includes subplots A-C, shows performance results of PIPI3 against other methods by using simulated tandem mass spectra datasets. Subplot A shows the numbers of PSMs with correct backbones (solid lines in the upper plot) and the number of PSMs with correct PTM patterns (dashed lines in the upper plot). The precision of PTM characterization is calculated by the number of PSMs with correct PTM patterns divided by the number of PSMs identified as carrying PTMs (lower plot). Subplot B shows the sensitivity of PTM characterization, calculated by the number of PSMs with correct PTM patterns divided by the ground truth number of MS2 spectra with PTMs. Subplot C shows the sensitivity of each individual PTM. As demonstrated in subplots A-C, on the simulation data sets with up to four PTMs per peptide, PIPI3 correctly identified over 99% of the spectra and characterized the PTM patterns with a precision of 85%, while the numbers of the best existing technique, MODplus, are 92% and 76%, highlighting PIPI3's advantage in handling peptides with multiple PTMs compared to state-of-the-art techniques. Real data examples also demonstrate that PIPI3 is an enabling tool to obtain a deeper insight into PTM combinations.

A second aspect of the present disclosure is to provide a system for identifying a plurality of peptides from a protein sample. Each peptide in the plurality of peptides is probable of having multiple PTMs. More specifically, each peptide may have any number of PTMs, including special cases of having no PTM and of having a single PTM. The disclosed system is intended to realize the method (or the workflow 100) disclosed above.

In the workflow 100, the steps 110, 120, 130, 140, 142, 144, 150 and 160 may be realized by a computer. The tandem mass spectra dataset to be obtained by the computer in the step 110 may be generated by a tandem mass spectrometer. The aforesaid arrangement is used in the development of the disclosed system. FIG. 4 depicts an exemplary system 400 for identifying the plurality of peptides from the sample (referenced as 480), where the system 400 is designed according to the aforesaid arrangement.

The system 400 comprises one or more computers 410 configured to execute a process of identifying the plurality of peptides from the protein sample 480 according to any of the embodiments of the disclosed method as exemplified by the workflow 100.

In certain embodiments, the system 400 further comprises a tandem mass spectrometer 420 for analyzing the sample 480 with tandem mass spectroscopy to thereby generate the tandem mass spectra dataset (referenced as 482) obtained for the sample 480. The tandem mass spectra dataset 482 generated by the tandem mass spectrometer 420 is sent to the one or more computers 410.

Regarding the one or more computers 410, an individual computer may be: a general-purpose computer; a special-purpose computer equipped with one or more specialized processors such as a graphics processing unit; a portable computer; a mobile computing device such as a smartphone; a computing server; a cloud server; or any computing device deemed appropriate by those skilled in the art.

The present disclosure may be embodied in other specific forms without departing from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

### REFERENCES

There follows a list of references that are occasionally cited in the specification. Each of the disclosures of these references is incorporated by reference herein in its entirety.
[1] Beausoleil S A, Villén J, Gerber S A, et al. A probability-based approach for high-throughput protein phosphorylation analysis and site localization [J]. Nature Biotechnology, 2006, 24(10): 1285-1292.
[2] Hunter T. The age of crosstalk: Phosphorylation, ubiquitination, and beyond [J]. Molecular Cell, 2007, 28(5): 730-738.
[3] Huang Y, Xu B, Zhou X, et al. Systematic characterization and prediction of post-translational modification cross-talk [J]. Molecular & Cellular Proteomics, 2015, 14(3): 761-770.
[4] Creasy D M, Cottrell J S. Unimod: Protein modifications for mass spectrometry [J]. Proteomics, 2004, 4(6): 1534-1536.
[5] Han X, He L, Xin L, et al. PeaksPTM: Mass spectrometry-based identification of peptides with unspecified modifications [J]. Journal of Proteome Research, 2011, 10(7): 2930-2936.
[6] Na S, Kim J, Paek E. MODplus: Robust and unrestrictive identification of post-translational modifications using mass spectrometry [J]. Analytical Chemistry, 2019, 91(17): 11324-11333.
[7] Yu F, Li N, Yu W. PIPI: PTM-invariant peptide identification using coding method [J]. Journal of Proteome Research, 2016, 15(12): 4423-4435.
[8] Chi H, Liu C, Yang H, et al. Comprehensive identification of peptides in tandem mass spectra using an efficient open search engine [J]. Nature Biotechnology, 2018, 36(11): 1059-1061.
[9] Devabhaktuni A, Lin S, Zhang L, et al. TagGraph reveals vast protein modification landscapes from large tandem mass spectrometry datasets [J]. Nature Biotechnology, 2019, 37(4): 469-479.
[10] Kong A T, Leprevost F V, Avtonomov D M, et al. MSFragger: Ultrafast and comprehensive peptide identification in mass spectrometry-based proteomics [J]. Nature Methods, 2017, 14(5): 513-520.
[11] Chambers M C, Maclean B, Burke R, et al. A cross-platform toolkit for mass spectrometry and proteomics [J]. Nature Biotechnology, 2012, 30(10): 918-920.
[12] Ferragina P, Manzini G. Opportunistic data structures with applications [C]//Proceedings 41 st Annual Symposium on Foundations of Computer Science. IEEE, 2000:390-398.
[13] Gurobi Optimization, LLC, “Gurobi Optimizer Reference Manual,” 2023. Accessed data: May 15, 2024.
[14] Zhou C. Dai S. Lin Y. et al. Exhaustive cross-linking search with protein feedback [J]. Journal of Proteome Research. 2022, 22(1): 101-113.
[15] Elias J E. Gygi S P. Target-decoy search strategy for increased confidence in large-scale protein identifications by mass spectrometry [J]. Nature Methods, 2007. 4(3): 207-214.

What is claimed is:

1. A method for identifying a plurality of peptides from a protein sample, an individual peptide in the plurality of peptides being probable of having multiple post-translational modifications (PTMs), the method comprising the steps of:
(a) obtaining a tandem mass spectra dataset obtained for the sample;
(b) extracting tags from a weighted directed graph formed according to spectral peaks identified in the dataset;
(c) using the extracted tags to retrieve a plurality of protein candidates potentially present in the sample from a FM-indexed protein database;
(d) segmenting an individual protein candidate into a plurality of sections according to locations of the extracted tags resided in the individual protein candidate, wherein the plurality of sections consists of a N-section, a C-section and a gap section;
(e) optimizing a mixed integer linear programming (MILP) model that models an individual section of the individual protein candidate to yield a peptide backbone and a PTM pattern for the individual section, wherein the MILP model is optimized under an objective of maximizing a total intensity of matched spectral peaks as matched by b- and y-ions in the individual section;
(f) repeating the steps (d) and (e) until the plurality of protein candidates is processed; and
(g) forming the plurality of peptides according to respective peptide backbones and respective PTM patterns obtained for the plurality of protein candidates.

2. The method of claim 1 further comprising the step (h) of using a target-decoy strategy to assess quality of the plurality of peptides obtained in the step (g).

3. The method of claim 2, wherein in the step (h), a false-discovery rate (FDR) is computed for the individual peptide according to the target-decoy strategy.

4. The method of claim 1, wherein in the step (c), the plurality of protein candidates is identified by using a fuzzy and bidirectional match strategy to match the extracted tags in respective peptide sequences stored in the FM-indexed protein database.

5. The method of claim 1, wherein in the step (b), the spectral peaks in the dataset are first identified according to a tandem mass spectral database.

6. The method of claim 1, wherein in the step (e), the MILP model is optimized by Gurobi Optimizer.

7. The method of claim 1, wherein in the step (b), the weighted directed graph is formed by calculating nodes and edges of the weighted directed graph according to the spectral peaks and potential amino acids that lie between successive peaks.

8. The method of claim 1, wherein in the step (c), the plurality of protein candidates is retrieved by using the extracted tags having various lengths.

9. The method of claim 8, wherein a FM-index used in the FM-indexed protein database consists of a forward index and a backward index such that bidirectional searches are supported.

10. The method of claim 9, wherein the FM-index is modified to enable a fuzzy search.

11. The method of claim 1, wherein in the step (d), amino acids in either the N-section or the C-section are grouped into fixed ones, optional ones and infeasible ones.

12. The method of claim 1, wherein constraints in the MILP model involve all fixed and optional amino acids, and all possible PTMs on every amino acid as recorded in the UNIMOD database.

13. A system for identifying a plurality of peptides from a protein sample, an individual peptide in the plurality of peptides being probable of having multiple post-translational modifications (PTMs), wherein the system comprises one or more computers configured to execute a process of identifying the plurality of peptides from the protein sample according to the method of claim 1.

14. The system of claim 13 further comprising a tandem mass spectrometer for analyzing the sample with tandem mass spectroscopy to thereby generate the tandem mass spectra dataset obtained for the sample.

15. A system for identifying a plurality of peptides from a protein sample, an individual peptide in the plurality of peptides being probable of having multiple post-translational modifications (PTMs), wherein the system comprises one or more computers configured to execute a process of identifying the plurality of peptides from the protein sample according to the method of claim 2.

16. The system of claim 15 further comprising a tandem mass spectrometer for analyzing the sample with tandem mass spectroscopy to thereby generate the tandem mass spectra dataset obtained for the sample.

17. A system for identifying a plurality of peptides from a protein sample, an individual peptide in the plurality of peptides being probable of having multiple post-translational modifications (PTMs), wherein the system comprises one or more computers configured to execute a process of identifying the plurality of peptides from the protein sample according to the method of claim 3.

18. The system of claim 17 further comprising a tandem mass spectrometer for analyzing the sample with tandem mass spectroscopy to thereby generate the tandem mass spectra dataset obtained for the sample.
